(19) United States
(12) Patent Application Publication
(10) Pub. No.: US 2004/0002710 A1
(43) Pub. Date: Jan. 1, 2004

(54) Ti-Ni-Mo shape memory alloy biomaterial and fixating device for bone fractures using the same alloy

(76) Inventors: Ki Suk Han, Ulsan (KR); Ji Soon Kim, Ulsan (KR); Seung Balk Kang, Seoul (KR); Tae Hyun Nam, Chinju (KR); Dong Geun Bark, Ulsan (KR)

Correspondence Address: Joseph W. Berenato, III, Liniak, Berenato, Longacre & White, LLC, Ste. 240, 6550 Rock Spring Drive, Bethesda, MD, 20817, US

(21) Appl. No.: 10/185,768

(22) Filed: Jul. 1, 2002

(51) Int. Cl. A61B 17/56

(52) U.S. Cl. 606/72

(57) ABSTRACT

The Ti—Ni—Mo shape memory alloy and fixating device for bone fractures using the same are provided, in which a very small amount of Mo of 0.5 at % or 0.7 at % is added Ni for to a Ti—Ni alloy, in order to maintain a transformation temperature whose martensite transformation start temperature (Rs) is 4-35° C. and whose inverse transformation finish temperature (Af) is 6-37° C. to be consistent, so that the transformation temperature can be applied to the human body most ideally, and enhance a corrosion resistivity. The Ti—Ni—Mo shape memory alloy is preferably made of Ti of 48-52 at %, Ni of 48-52 at % and Mo of 0.1-2.0 at %, in a composition ratio. In the case of a B2 (Cubic)⇄R (Rhombohedral)⇄B19′(Monoclinic) transformation, the Ti—Ni—Mo shape memory alloy reduces a variation in a transformation start temperature and an inverse transformation finish temperature according to an annealing temperature change, to thus maintain the transformation temperature constantly. Also, the Ti—Ni—Mo shape memory alloy possesses the most appropriate transformation temperature to be applied to the human body and an enhanced corrosion resistivity when an amount of Mo added is increased, and reduces Ni dissolution quantity as can be seen from Ni dissolution test to thereby enhance biocompatibility in the human body.

### BACKGROUND OF THE INVENTION

1. Field of the Invention

The present invention relates to Ti—Ni—Mo shape memory alloy biomaterial and a fixating device for bone fractures using the same alloy biomaterial, in which transformation temperature can be constantly maintained so as to be applied to the human body most ideally, and a Ni dissolution quantity is reduced due to an increased corrosion resistivity, and more particularly, to a Ti—Ni—Mo shape memory alloy and a fixating device for bone fractures using the same alloy biomaterial, in which Mo is added in a Ti—Ni shape memory alloy to thus consistently maintain a transformation temperature even with an annealing temperature change.

2. Description of the Related Art

In general, a shape memory alloy is classified into a Ti—Ni alloy, a Cu alloy, and a Fe alloy, among which the Ti—Ni alloy is most widely used.

However, a Ti—Ni alloy having an equiatomic ratio composition is known as a B2 (Cubic)⇄B19′ (Monoclinic) transformation material. In the case that the Ti—Ni alloy is thermally treated or added with a third element such as Al and Fe, it is known to become a B2 (Cubic)⇄R (Rhombohedral)⇄B19′(Monoclinic) transformation material.

A transformation deformation ratio and a transformation histeresis accompanying a B2⇄R transformation are very small as 0.8% and 2K, respectively.

Meanwhile, in order to make an alloy operate accurately at designed temperature when a Ti—Ni shape memory alloy is used as a driving device, martensite transformation start temperature (Ms or T_{R}) and an inverse transformation finish temperature (Af) in the alloy should equal the designed temperature. However, since the transformation temperature of a conventional Ti—Ni shape memory alloy varies very sensitively according to the composition, processing, thermal treatment, use condition of the alloy, and so on, these variables should be controlled accurately during manufacturing in order to make the designed temperature and the transformation temperature equal.

Also, in order to actually use the Ti—Ni shape memory alloy, the alloy should be processed as a plate material or a linear material. However, since the Ti—Ni alloy shape memory alloy has a very high work hardening constant, an annealing treatment should be performed during manufacturing in order to lower an internal stress.

As described above, when the annealing treatment is performed after a cold working, the transformation temperature is greatly varied according to the annealing temperature and time.

In particular, it has been reported that in the case of a B2⇄B19′ transformation which is the inverse transformation, the transformation temperature change results in about 40K, and in the case of a B2 (Cubic)⇄R (Rhombohedral)⇄B19′(Monoclinic) transformation, the temperature change results in about 20K.

This is because R phase and B19′ phase appear in an overlapping pattern at the time of an inverse transformation since a stability in the R phase is inferior in the case of an alloy inducing a conventional R phase transformation.

As described above, since the transformation temperature greatly changes with respect to a change in an annealing processing condition, a very precise and complicated manufacturing process is required in order to satisfy a temperature required as a driving device in a system.

Meanwhile, in the case that a metal material is implanted into a living body and then used functionally, a biocompatibility with respect to the metal material should be considered. In general, there are a corrosion resistivity test, a cell cultivation test and an animal test as a biocompatibility estimation method of the Ti—Ni shape memory alloy.

Among the biocompatibility estimation methods, the corrosion resistivity test functions as an important factor. This is because metal may be harmful in the case that the metal is dissolved as ions at a corrosion environment in the human body.

In particular, nickel is known as a harmful element for the human body. Since the Ti—Ni shape memory alloy is made of Ni of 45-55 at %, it is an important issue whether or not the Ti—Ni shape memory alloy can be applied as an organism material. Accordingly, a research for reducing a Ni dissolution quantity is proceeding.

Thus, in order to use the conventional Ti—Ni shape memory alloy as a further more stable biomaterial, a corrosion property is enhanced to thereby make a Ni dissolution quantity small.

As described above, the conventional Ti—Ni shape memory alloy has problems that the transformation temperature greatly varies according to variation in an annealing treatment condition, and metal is harmful with respect to the human body in the case that metal is corroded and dissolved as ions.

In the result of a research for solving the above problems, the inventors found that the Ti—Ni—Mo alloy which can maintain uniformity of the transformation temperature even with a variation in an annealing treatment condition, can be obtained by adding Mo in the Ti—Ni alloy to induce a R phase and enhanced a stability of the produced R phase.

And, when Mo is added into a Ti—Ni—Mo alloy, the corrosion resistivity is increased and thus the Ti—Ni—Mo shape memory alloy whose Ni dissolution quantity is reduced can be obtained.

### SUMMARY OF THE INVENTION

To solve the above problems, it is an object of the present invention to provide a shape memory alloy having the constant transformation temperature although an annealing treatment condition is varied, in which Mo is added into the Ti—Ni—Mo alloy.

It is another object of the present invention to provide a Ti—Ni—Mo shape memory alloy which is appropriate for a biomaterial in which Mo is added into a Ti—Ni alloy, to thereby increase the corrosion resistivity to thus reduce Ni dissolution quantity.

It is still another object of the present invention to provide a fixating device for bone fractures using the Ti—Ni—Mo shape memory alloy.

To accomplish the above object of the present invention, there is provided a Ti—Ni—Mo shape memory alloy made of Ti of 48-52 at %, Ni of 48-52 at %, and Mo of 0.1-2.0 at % in a composition ratio.

Also, a fixating device for bone fractures using the Ti—Ni—Mo shape memory alloy can be applied through a smaller incision and opening, in comparison with a conventional surgical operation. Also, the fixating device for bone fractures can be easily applied, shortens an operation time and induces an earlier recovery of a patient after surgical operation.

### BRIEF DESCRIPTION OF THE DRAWINGS

The above objects and other advantages of the present invention will become more apparent by describing the preferred embodiments thereof in more detail with reference to the accompanying drawings in which:

FIG. 1A is a graph showing differential scanning calorimetry (DSC) curves when an annealing treatment of the Ti—Ni alloy which is a 51Ti—49Ni at % alloy is performed after a cold working according to a conventional comparative example, and FIG. 1B is a graph showing DSC curves when an annealing treatment of the Ti—Ni alloy which is a 51Ti—49Ni at % alloy is performed after a heat working according to the conventional comparative example;

FIG. 2 is a graph showing variation in a transformation start temperature (Ms or T_{R}) and an inverse transformation finish temperature (Af) according to an annealing temperature change in the 51Ti—49Ni at % alloy of the conventional comparative example;

FIG. 3A is a graph showing DSC curves when an annealing treatment of the Ti—Ni—Mo alloy which is the 51Ti—48.5Ni—0.5Mo at % alloy is performed after a cold working according to a second embodiment of the present invention, and FIG. 3B is a graph showing DSC curves when an annealing treatment of the Ti—Ni—Mo alloy which is the 51Ti—48.5Ni—0.5Mo at % alloy is performed after a heat working according to the second embodiment of the present invention;

FIG. 4 is a graph showing variation in a transformation start temperature (Ms or T_{R}) and an inverse transformation finish temperature (Af) according to the annealing temperature change in the 51Ti—48.5Ni—0.5Mo at % alloy according to the second embodiment of the present invention;

FIG. 5A is a graph showing DSC curves when an annealing treatment of the Ti—Ni—Mo alloy which is the 51Ti—48.3Ni—0.7Mo at % alloy is performed after a cold working according to a third embodiment of the present invention, and FIG. 5B is a graph showing DSC curves when an annealing treatment of the Ti—Ni—Mo alloy which is the 51Ti—48.3Ni—0.7Mo at % alloy is performed after a heat working according to a third embodiment of the present invention;

FIG. 6 is a graph showing variation in a transformation start temperature (Ms or T_{R}) and an inverse transformation finish temperature (Af) according to an annealing temperature change in the 51Ti—48.3Ni—0.7Mo at % alloy according to the third embodiment of the present invention;

FIG. 7A is a graph showing dependency of Mo upon variation in a transformation start temperature (Ms or T_{R}) according to an annealing temperature change in the Ti—Ni—Mo alloy according to the present invention, and FIG. 7B is a graph showing dependency of Mo upon variation in an inverse transformation finish temperature (Af) according to an annealing temperature change in the Ti—Ni—Mo alloy according to the present invention.;

FIG. 8 is a graph showing a potentio-dynamic polarization test result for grasping a corrosion resistivity of the 51Ti—49Ni at % alloy of the conventional comparative example;

FIG. 9 is a graph showing a potentio-dynamic polarization test result for grasping a corrosion resistivity of the 51Ti—48.5Ni—0.5Mo at % alloy according to the second embodiment of the present invention;

FIG. 10 is a graph showing a potentio-dynamic polarization test result for grasping a corrosion resistivity of the 51Ti—48.3Ni—0.7Mo at % alloy according to the third embodiment of the present invention;

FIGS. 11A and 11B show a single ring type fixating device for bone fractures using the Ti—Ni—Mo shape memory alloy, respectively;

FIGS. 12A and 12B show a double ring type fixating device for bone fractures using the Ti—Ni—Mo shape memory alloy, respectively;

FIGS. 13A1, 13A2, 13A3, 13B and 13C show a long leg omega type fixating device for bone fractures using the Ti—Ni—Mo shape memory alloy, respectively;

FIGS. 14A1, 14A2, 14A3, 14B1, 14B2 and 14C show an omega ring type fixating device for bone fractures using the Ti—Ni—Mo shape memory alloy, respectively;

FIGS. 15A, 15B and 15C show an ellipse type fixating device for bone fractures using a Ti—Ni—Mo shape memory alloy, respectively;

FIGS. 16A, 16B and 16C show a clip type fixating device for bone fractures using the Ti—Ni—Mo shape memory alloy, respectively;

FIGS. 17A1, 17A2, 17A3, 17B1, and 17B2 show a wave ring type fixating device for bone fractures using the Ti—Ni—Mo shape memory alloy, respectively;

FIGS. 18A1, 18A2, 18A3, and 18B show a multi-omega ring type fixating device for bone fractures using the Ti—Ni—Mo shape memory alloy, respectively; and

FIGS. 19A1, 19A2, 19A3, 19B1, and 19B2 show an omega type fixating device for bone fractures using the Ti—Ni—Mo shape memory alloy, respectively.

### DETAILED DESCRIPTION OF THE INVENTION

Preferred embodiments of the present invention will be described with reference to the accompanying drawings.

First, the 51Ti—48.5Ni—0.5Mo (at %) alloy according to a second embodiment of the present invention is manufactured by substitution Ni for Mo of 0.5 at % into the 51Ti—49Ni (at %) shape memory alloy.

Since a melting point of Mo is very high as 2610° C., a master alloy of Ti(Ni) and Mo is manufactured by using the plasma melting method during manufacturing an alloy. Then, the manufactured master alloy, sponge Ti (purity 99.6%), electrolytic Ni (purity 99.9%) are introduced into a graphite furnace and then melted at a high frequency induction melting furnace in vacuum.

A manufactured ingot is hot rolled at 1123K, and then cold worked as a wire of 1.2 mm in diameter at 298K. Here, a cold working ratio is made 25%.

The following Table 1 illustrates quantities of Ti, Ni and Mo applied in a shape memory alloy according to the present invention. First embodiment, and third through sixth embodiments according to the present invention provide a shape memory alloy manufactured in the same manner as that of the second embodiment, except for quantities of Ti, Ni and Mo illustrated in the following Table 1.

TABLE 1

| Composite | | | | | | | |
|---|---|---|---|---|---|---|---|
| element | 1st | 2nd | 3rd | 4th | 5th | 6th | comparative |
| (at %) | embodiment | embodiment | embodiment | embodiment | embodiment | embodiment | example |
| Ti | 51 | 51 | 51 | 51 | 51 | 51 | 51 |
| Ni | 48.9 | 48.5 | 48.3 | 48 | 47.5 | 47 | 49 |
| Mo | 0.1 | 0.5 | 0.7 | 1.0 | 1.5 | 2 | |

A conventional 51Ti—49Ni (at %) shape memory alloy has been manufactured as a first comparative example, for comparison with a Ti—Ni—Mo alloy according to the present invention.

The 51Ti—49Ni (at %) shape memory alloy has been manufactured by introducing sponge Ti (purity 99.6%), electrolytic Ni (purity 99.9%) into a graphite crucible and then melting them at high frequency induction melting furnace in vacuum. A manufactured ingot is hot rolled at 1123K, and then cold worked as a wire of 1.2 mm in diameter at 298K. Here, a cold working ratio is made 25%.

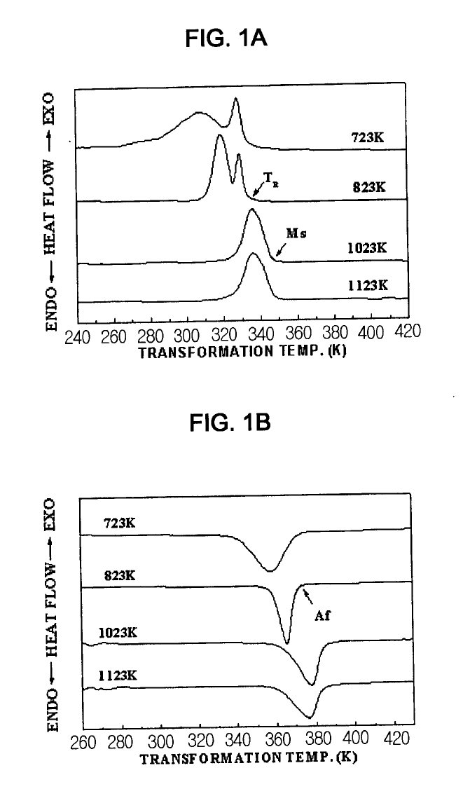

FIG. 1A is a graph showing differential scanning calorimetry (DSC) curves when an annealing treatment of the Ti—Ni alloy which is a 51Ni—49Ni at % alloy is performed after a cold working according to a conventional comparative example, and FIG. 1B is a graph showing DSC curves when an annealing treatment of the Ti—Ni alloy which is a 51Ti—49Ni at % alloy is performed after a heat working according to the conventional comparative example.

As can be seen from FIG. 1A, when an annealing temperature reaches 723K, two heat emission peaks are observed during cooling, in which high temperature peak corresponds the B2→R transformation, and a low temperature peak corresponds to the R→B19′ transformation. And, when the annealing temperature is higher than 1023K, only one peak overlaps. As can be seen from FIG. 1B, only one peak is observed during heating. This is because the R→B2 transformation and the B19′→R transformation overlap with each other since a stability on the R phase is inferior.

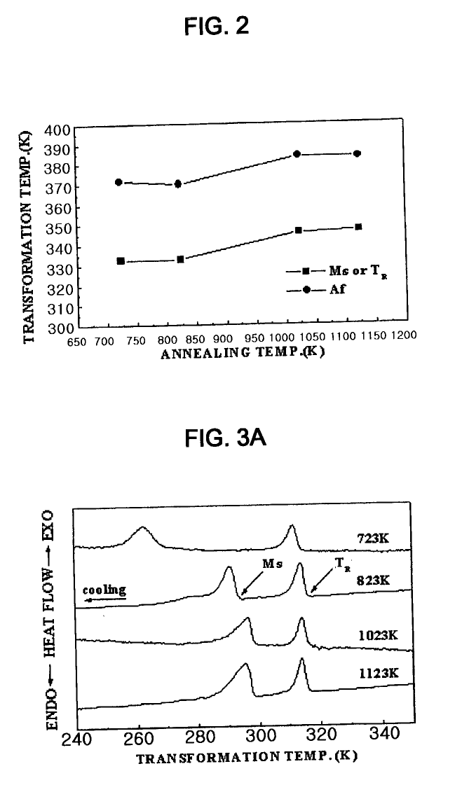

FIG. 2 is a graph showing variation in the transformation start temperature (Ms or T_{R}) and inverse transformation finish temperature (Af) according to an annealing temperature change in the 51Ti—49Ni at % alloy manufactured by the conventional comparative example.

As can be seen from FIG. 2, when an annealing temperature rises up from 723K to 1123K, the transformation start temperature (Ms or T_{R}) varies by about 15K, and the inverse transformation finish temperature varies by about 13K.

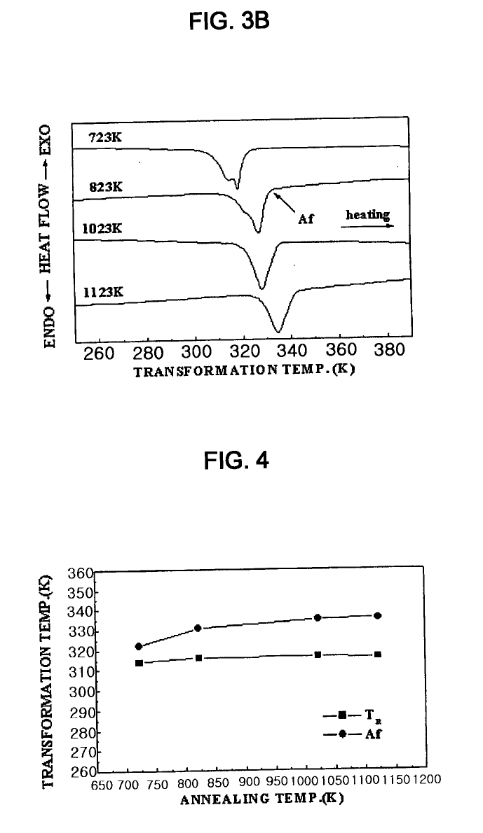

FIG. 3A is a graph showing DSC curves when an annealing treatment of the Ti—Ni—Mo alloy which is the 51Ti—48.5Ni—0.5Mo at % alloy is performed after a cold working according to a second embodiment of the present invention, and FIG. 3B is a graph showing DSC curves when an annealing treatment of the Ti—Ni—Mo alloy which is the 51Ti—48.5Ni—0.5Mo at % alloy is performed after a heat working according to the second embodiment of the present invention.

As can be seen from FIG. 3A, two heat emission peaks are observed at all annealing temperatures during cooling.

However, as can be seen from FIG. 3B, although two heat absorption peaks are observed in the case that annealing temperature is 723K and 823K during heating, the two peaks are not completely separated from each other. Only one peak is observed in the case that an annealing temperature is more than 823K.

FIG. 4 is a graph showing variation in the transformation start temperature (Ms or T_{R}) and inverse transformation end temperature (Af) according to an annealing temperature change in the 51Ti—48.5Ni—0.5Mo at % alloy according to the second embodiment of the present invention.

As can be seen from FIG. 4, when an annealing temperature rises up from 673K to 1123K, the transformation start temperature (Ms) varies by about 2K, and the inverse transformation finish temperature varies by about 12K.

When the above-described results are compared with the 51Ti—49Ni at % alloy according to the conventional comparative example, added Mo into a two-element alloy of Ti—Ni results in enhancement of a stability of R phase. As a result, it can be seen that variation in the transformation start temperature (Ms or T_{R}) and the inverse transformation finish temperature (Af) according to an annealing temperature change is reduced.

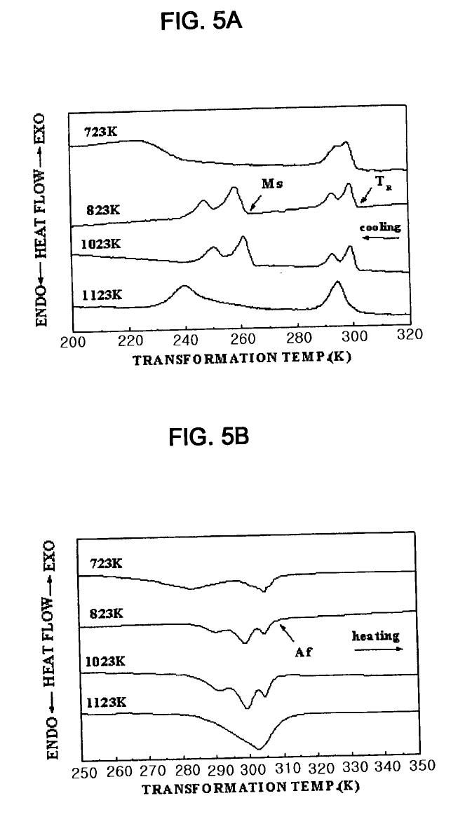

FIG. 5A is a graph showing DSC curves when an annealing treatment of the Ti—Ni—Mo alloy which is the 51Ni—48.3Ni—0.7Mo at % alloy is performed after a cold working according to a third embodiment of the present invention, and FIG. 3B is a graph showing DSC curves when an annealing treatment of the Ti—Ni—Mo alloy which is the 51Ti—48.3Ni—0.7Mo at % alloy is performed after a heat working according to a third embodiment of the present invention.

As can be seen from FIG. 5A, two or more heat emission peaks are observed at all annealing temperatures.

It can be also seen from FIG. 5B that two or more heat emission peaks are observed at all annealing temperatures during heating. This means that B19′→R transformation and R→B2 transformation do not overlap with each other and completely separated.

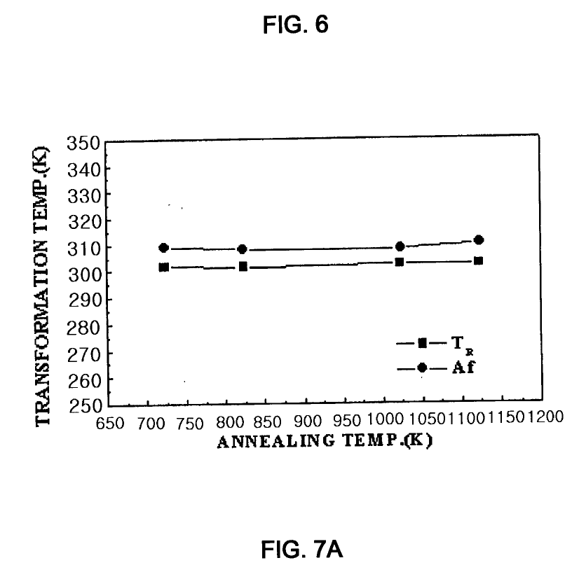

FIG. 6 is a graph showing variation in the transformation start temperature (Ms or T_{R}) and inverse transformation finish temperature (Af) according to an annealing temperature change in the 51Ti—48.3Ni—0.7Mo at % alloy according to the third embodiment of the present invention.

As can be seen from FIG. 6, in the case of the 51Ni—48.3Ni—0.7Mo at % alloy according to the third embodiment of the present invention, it can be seen that variation in the transformation start temperature (Ms or T_{R}) and inverse transformation finish temperature (Af) according to an annealing temperature change is very small as less than 2K.

When these results are compared with the FIG. 2 result according to the comparative example and the FIG. 4 result according to the third embodiment of the present invention, addition of Mo results in an increase in a stability. Accordingly, since R phase is separated from B19′ phase in the case of an inverse transformation, it can be seen that an inverse transformation temperature change is much smaller than that of the case that Mo is not added.

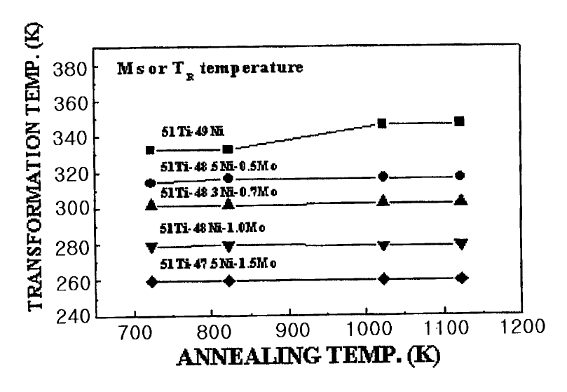

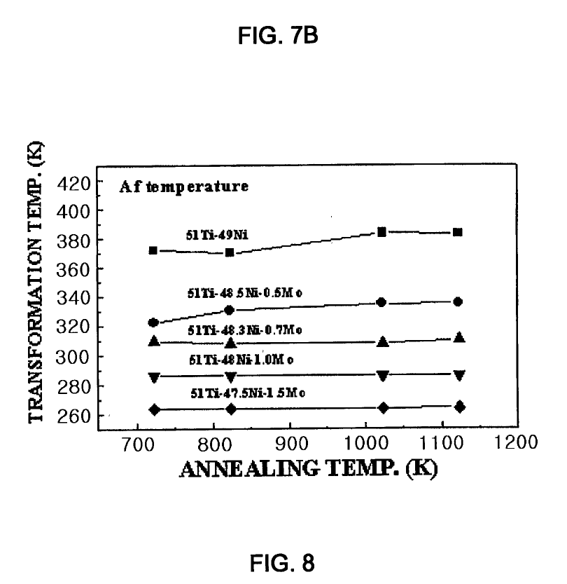

FIGS. 7A and 7B are graphs showing variation in the transformation start temperature (Ms or T_{R}) and an inverse transformation finish temperature (Af) of martensite according to an annealing temperature change during annealing after cold working in the Ti—Ni alloy according to the conventional comparative example and the Ti—Ni—Mo alloys according to the first through fifth embodiments of the present invention.

That is, FIGS. 7A and 7B are graphs showing dependency of Mo upon variation in the transformation start temperature (Ms or T_{R}) and inverse transformation finish temperature (Af) according to an annealing temperature change in the Ti—Ni—Mo alloy according to the present invention.

As can be seen from FIG. 7A, it can be seen that the variation in the transformation start temperature is decreased, as Mo is added from the Ti—Ni—Mo alloy, but the variation in the transformation start temperature is not nearly noted in the case of Mo of 0.5 t % or higher.

Thus, as can be seen in FIG. 7B, it can be seen that the variation in the inverse transformation finish temperature is decreased, as Mo is added and is very small as not more than 2K in the case of Mo of 0.7 at % or higher.

Therefore, when Mo is added into a Ti—Ni shape memory alloy, a stability on R phase is increased. As a result, it can be seen that the inverse transformation finish temperature change can be made very small upon an annealing treatment condition (temperature) during annealing after cold working. That is, since the Ti—Ni—Mo shape memory alloy according to the present invention enhances R phase stability by adding Mo into the Ti—Ni alloy, the transformation temperature can be constantly maintained although the annealing condition is varied during annealing.

Also, when Mo is added into the Ti—Ni alloy, the corrosion resistivity is increased and Ni dissolution quantity is reduced. The thus-obtained Ti—Ni—Mo shape memory alloy will be described below.

FIGS. 8 through 10 show potentio-dynamic polarization test results for grasping a corrosion resistivity of the conventional comparative example and the second and third embodiments according to the present invention, respectively.

Here, an estimation method of a corrosion resistivity test is performed based on ASTM G5 (1994). The corrosion resistivity becomes high as potential becomes high.

FIG. 8 is a graph showing a potentio-dynamic polarization test result for grasping a corrosion resistivity of the 51Ti—49Ni at % alloy of the conventional comparative example.

As can be seen from FIG. 8, a current density is sharply increased in the vicinity of a pitting potential of 250 mV which indicates a corrosion resistivity.

The increase in the current density indicates that a corrosion of the 51Ti—49Ni at % alloy occurs at the pitting potential (corrosion resistivity) of 250 mV.

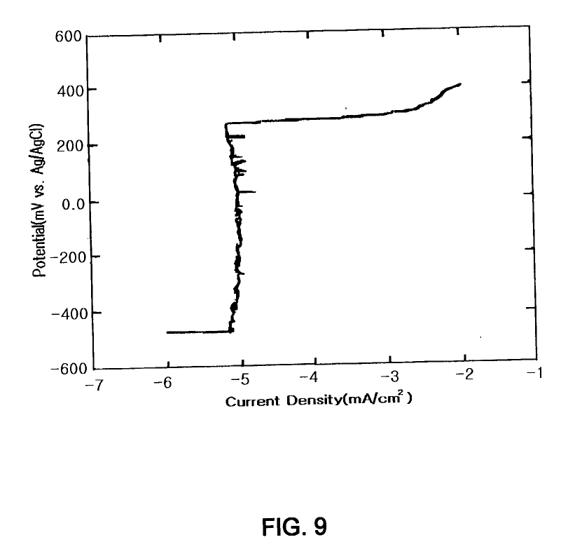

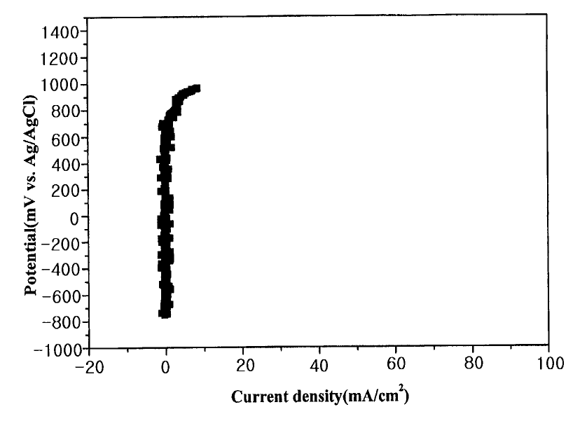

FIG. 9 is a graph showing a potentio-dynamic polarization test result for grasping a corrosion resistivity of the 51Ti—48.5Ni—0.5Mo at % alloy according to the second embodiment of the present invention.

As can be seen from FIG. 9, a current density is sharply increased in the vicinity of a pitting potential of 750 mV which indicates a corrosion resistivity.

The increase in the current density indicates that a corrosion of the 51Ti—48.5Ni—0.5Mo at % alloy occurs at the pitting potential (corrosion resistivity) of 750 mV.

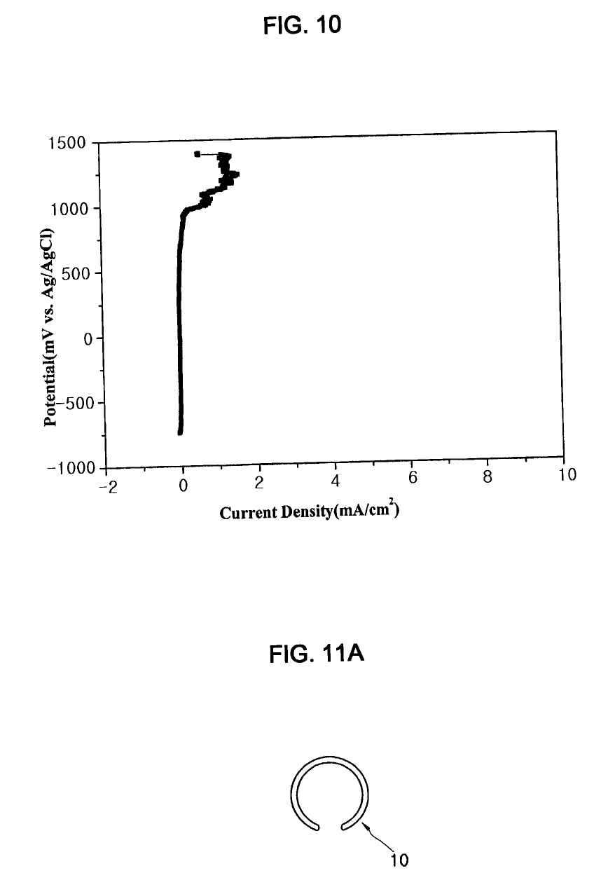

FIG. 10 is a graph showing a potentio-dynamic polarization test result for grasping a corrosion resistivity of the 51Ni—48.3Ni—0.7Mo at % alloy according to the third embodiment of the present invention.

As can be seen from FIG. 10, a current density is sharply increased in the vicinity of a pitting potential of 100 mV which indicates a corrosion resistivity.

The increase in the current density indicates that a corrosion of the 51Ni—48.3Ni—0.7Mo at % alloy occurs at the pitting potential (corrosion resistivity) of 1000 mV.

As can be seen from FIGS. 8 through 10, when Mo is added Mo into a Ti—Ni shape memory alloy, a pitting potential, that is, a corrosion resistivity is increased. It can be seen that the increase in the corrosion resistivity is increased as a Mo content is increased from 0 up to 0.5 and 0.7.

A Ti—Ni—Mo shape memory alloy whose Ni eruption quantity is reduced as Mo is added into a Ti—Ni alloy will be described below.

The following Table 2 illustrates dissolution test results of Ni.

TABLE 2

| Alloy composition | Ni dissolution quantity (mg/L) |
|---|---|
| 51Ti-49Ni (at %) | 0.107 |
| 51Ti-48.5Ni-0.5Mo (at %) | 0.044 |
| 51Ti-48.3-0.75Mo (at %) | 0.01 |

The 51Ni—49Nio at % according to the conventional comparative example, the 51Ti—48.5Ni—0.5Mo at % according to the second embodiment of the present invention, and the 51Ti—48.3Ni—0.7Mo at % according to the third embodiment of the present invention are put into a test bottle with 0.2 g/ml which is a ratio of a weight and a physiological saline solution of 0.9% NaCl, and then kept in a constant temperature bath for 72±2 hours at 50±2° C. Thereafter, the physiological saline solution is collected and then Ni dissolved in the physiological saline solution is ICP-analyzed to measure Ni dissolution quantity.

As can be seen from Table 2, the Ni dissolution quantity is reduced as a content of Mo is increased from 0 up to 0.5 and 0.7 at %.

From the above-described results, when Mo is added into the Ti—Ni shape memory alloy, it can be seen that a corrosion resistivity is enhanced and toxicity due to the Ni dissolution quantity is reduced.

FIGS. 1A through 19B2 show examples applied to fracture of a bone, which relate to a fixating device for bone fractures made of a Ti—Ni—Mo shape memory alloy as an example, respectively.

A conventional fixating device for bone fractures which is used for fracture of a bone wraps a fracture portion by using a steel single- or multiple-wire, or fixes the fracture portion with a metal plate by using screw bolts, clips or staples. During treatment of the conventional fixating device for bone fractures, a large-area incision and a broad opening of a fracture portion are inevitable. Also, a loss of a normal portion is unavoidable as in the case that holes are drilled onto a bone. Also, since a treatment method is difficult and much time is consumed, a surgical operation time becomes longer. On the contrary, the present invention can be applied through a small-area incision because of the feature of a shape memory alloy. Also, since a treatment is very easy, a surgical operation time can be shortened and an earlier recovery of a patient can be accomplished after surgical operation.

Since bones in the human body have various sectional shapes such as an ellipse, triangles, rectangles and so on, the present invention provides a shape memory alloy fixating device for bone fractures so that a high tensile stress can be maintained without harming a normal portion according to the shape of a bone.

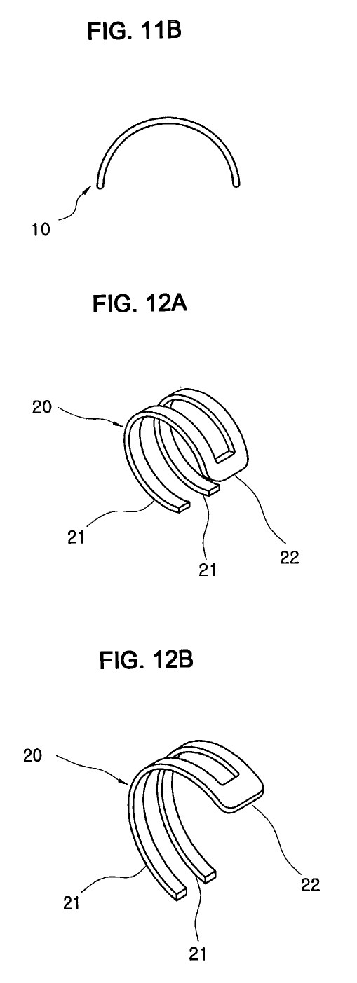

FIGS. 11A and 11B show a single ring type fixating device for bone fractures using a Ti—Ni—Mo shape memory alloy, respectively.

FIG. 11A shows a single ring type 10 memorizing a ring shape as a memorized shape. FIG. 11B shows a shape obtained by deforming the FIG. 11A single ring at a low temperature so that it can be easily applied to a fracture portion. Thus, if the ring type fixating device for bone fractures is applied to the fracture portion at a low temperature, it is recovered into the original shape of the FIG. 11A shape at the bodily temperature.

FIGS. 12A and 12B show a double ring type fixating device for bone fractures using a Ti—Ni—Mo shape memory alloy, respectively.

FIG. 12A shows a memorized shape and FIG. 12B shows a shape obtained by deforming the FIG. 12A shape at a low temperature.

As shown in FIGS. 12A and 12B, wires are rolled and worked in a rod shape in order to widen a contact area to a bone, and then fabricated into a double ring 20. Since the length of the junction is lengthy and the thickness thereof is thick, the double ring type fixating device for bone fractures is used when a strong tightening is needed.

Each holder 21 of the double ring is integrally formed with respect to a connection 22 so that fracture of a bone can be connected and then fixed.

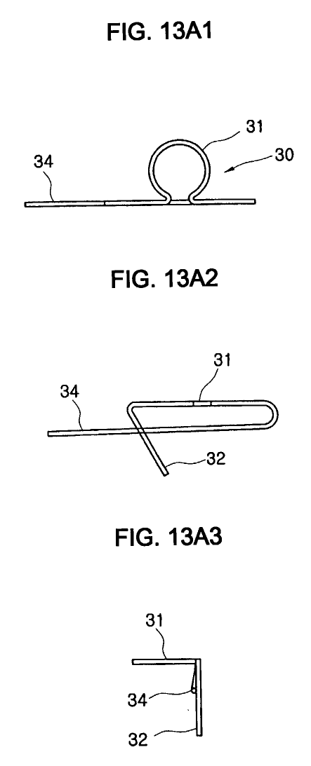

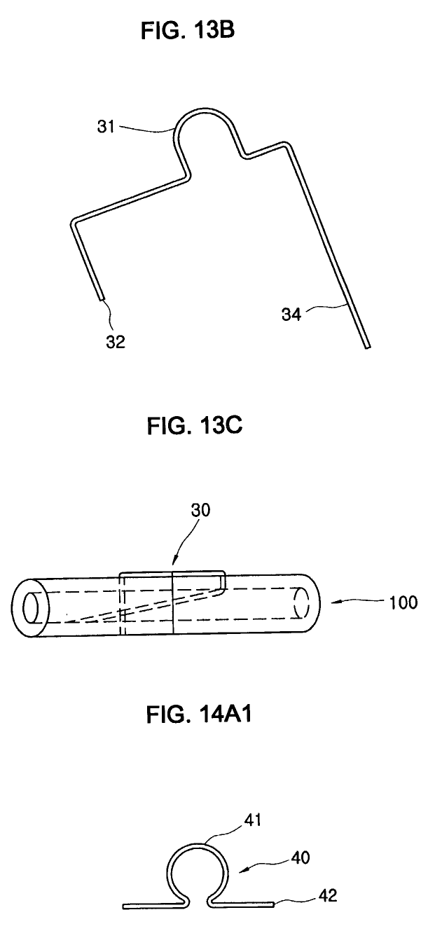

FIGS. 13A1, 13A2, 13A3, 13B and 13C show a long leg omega type fixating device for bone fractures using a Ti—Ni—Mo shape memory alloy, respectively.

FIGS. 13A1, 13A2 and 13A3 show a memorized shape, in which FIG. 13A1 is a plan view, FIG. 13A2 is a front view, and FIG. 13A3 is a side view. FIG. 13B shows a shape obtained by deforming the FIG. 13A1 shape at a low temperature, and FIG. 13C shows a state where the FIG. 13A1 fixating device for bone fractures is applied onto a fracture portion.

As shown in FIGS. 13A1, 13A2 and 13A3, the memorized shape forms an single ring 31 in the middle portion, and both ends 32 and 34 of the ring 31 are extended lengthily and then bent. Thereafter, both the ends 32 and 34 are crossed at the lower side of the ring 31.

FIG. 13B shows a shape obtained by deforming the ring 31 at a low temperature, which shows an extended state. FIG. 13C shows a state where the FIG. 13B fixating device for bone fractures is applied to a fracture portion. The short leg 32 is inserted into a hole obtained by drilling and penetrating a fracture bone 100, and a long leg 34 is inserted into another hole obtained by drilling and penetrating the upper horny bone and made to contact the inner wall of the lower horny bone. Then, the left and right portions of the ring pull both ends of the ring so that the fracture portion is not widened.

Since the inner portions of bones are hollow tubes, the present invention product includes a short leg and a long leg which transverse the center of the bone. Here, the short leg 32 and both ends of the ring 31 play a role of making the bone not move in the left and right directions, and the long leg 34 functions as a stable fixture by making the bone not move in the top and bottom direction.

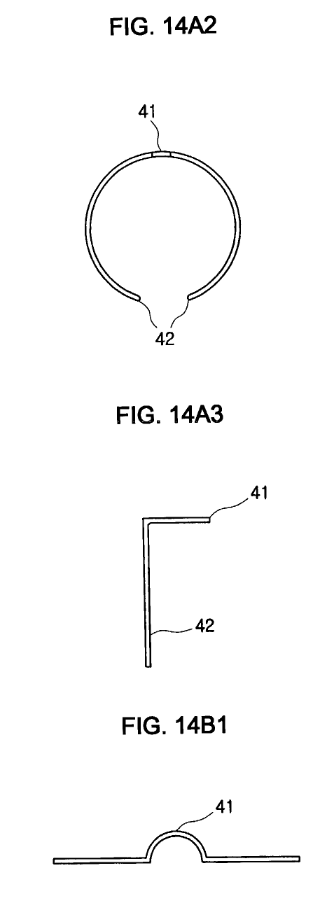

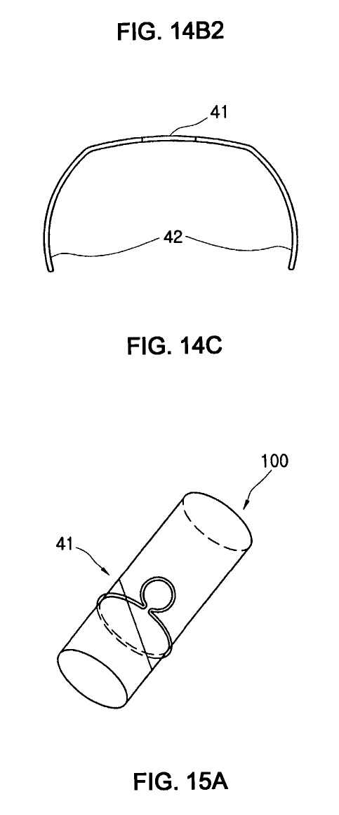

FIGS. 14A1, 14A2, 14A3, 14B1, 14B2 and 14C show an omega ring type fixating device for bone fractures using a Ti—Ni—Mo shape memory alloy, respectively.

FIGS. 14A1, 14A2 and 14A3 show a memorized shape, in which FIG. 14A1 is a plan view, FIG. 14A2 is a front view, and FIG. 14A3 is a side view. FIGS. 14B1 and 14B2 show a shape obtained by deforming the FIG. 14A1 shape at a low temperature, in which FIG. 14B1 is a plan view and FIG. 14B2 is a front view and FIG. 14C shows a state where the FIG. 14A1 fixating device for bone fractures is applied onto a fracture portion.

FIGS. 14A1, 14A2, 14A3, 14B1, 14B2 and 14C show an osseous junction instrument which can be easily fixed in the case that the shape of a bone is a circle, an ellipse, or the upper and lower portions of a bone are different in a surface area.

As shown in FIGS. 14A1, 14A2 and 14A3, the fixating device for bone fractures according to the present invention includes a ring 41 which can provide a fixing force in the left and right directions and a holder 42 which can wrap a bone.

As shown in FIGS. 14B1 and 14B2, the ring 41 and the holder 42 are widened laterally at a low temperature, so that an elliptical fracture portion can be wrapped and easily fixed.

As shown in FIG. 14C, the ring 41 shrinks to thus pull the holder 42 and the holder 42 wraps a fracture portion 100.

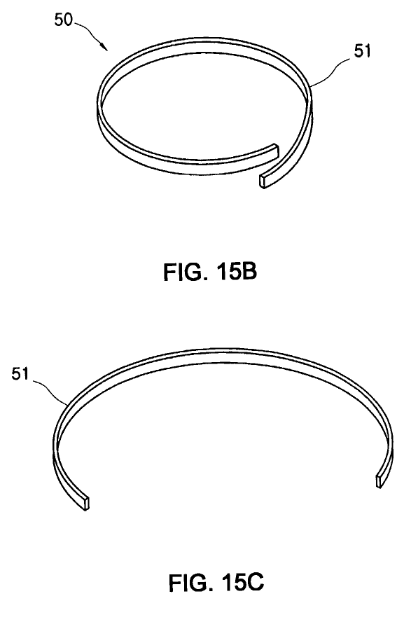

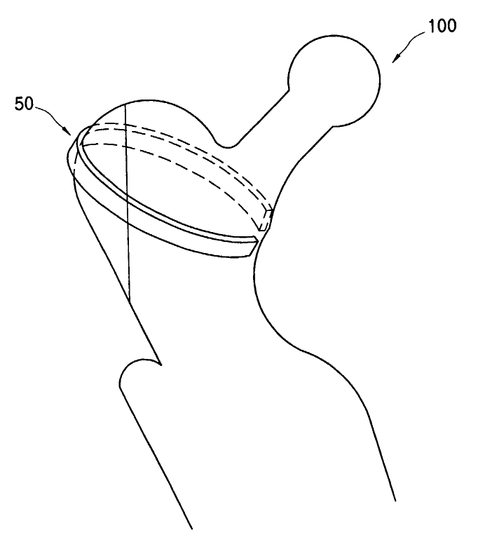

FIGS. 15A, 15B and 15C show an ellipse fixating device for bone fractures using a Ti—Ni—Mo shape memory alloy, respectively.

As shown in FIG. 15A, a fixating device for bone fractures is an elliptical clamp 51 in which both ends are crossed and a wire is rolled and fabricated in an elliptical shape in order to widen a contact area to a bone.

FIG. 15B shows a state where the clamp 51 is extended at a low temperature, and FIG. 15C shows a state where the FIG. 15B elliptical fixating device for bone fractures is fixed onto a fracture portion of the head of a bone in femur 100.

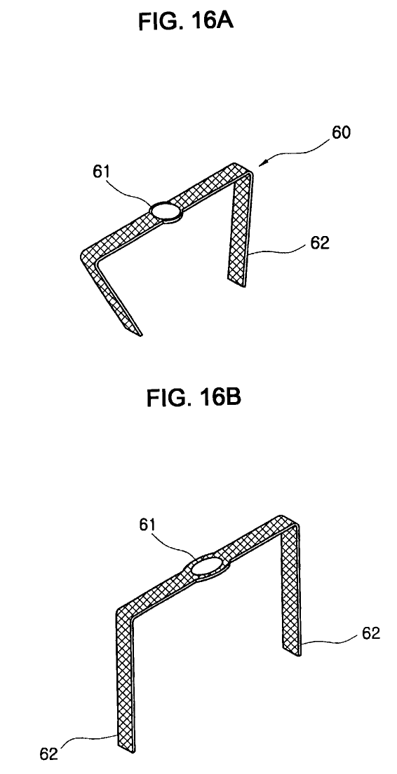

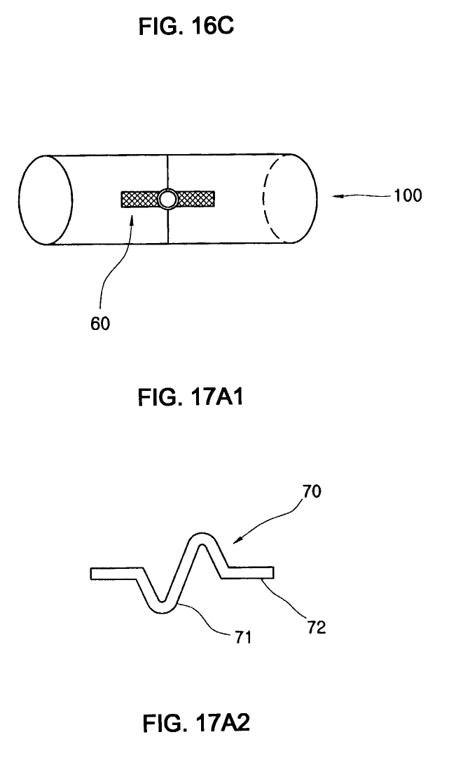

FIGS. 16A, 16B and 16C show a clip type fixating device for bone fractures using the Ti—Ni—Mo shape memory alloy, respectively.

As shown in FIG. 16A, two wires are collected and widened in both sides to make a single wire, which includes a ring type 61 and a plate type holder 62 that makes the wires twisted integrally or rolled.

The holder 62 is extended in both sides of the ring 61, bent in the middle portion and located with a predetermined angle with respect to the ring 61.

FIG. 16B shows a state where the ring 61 is deformed as an ellipse, and the holder 62 is bursted open so that the holder 62 is perpendicular with the ring 61.

In FIG. 16C, the holder 62 penetrates a bone 100 and tightens a fracture portion at both sides. The ring 61 is recovered into an original shape to thus pull and fasten the holder 62.

The clip type fixating device for bone fractures is apt to fail in exhibiting a fastening force of the holder 62, in the case that the ring 61 is made of a single wire.

Thus, to exhibit a fastening force effectively at the time of fastening a fracture portion, the fixating device for bone fractures should be made of two or more wires firmly so that a ring forming the center of the fixating device for bone fractures can support a holder.

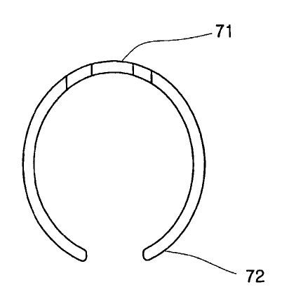

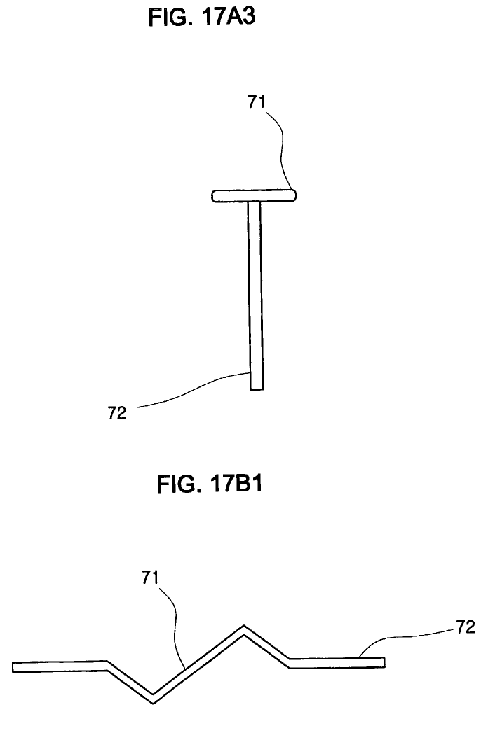

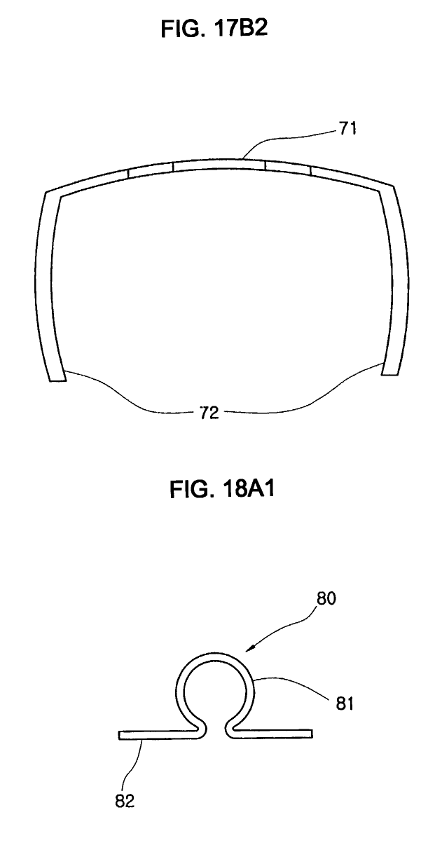

FIGS. 17A1, 17A2, 17A3, 17B1, and 17B2 show a wave ring type fixating device for bone fractures using a Ti—Ni—Mo shape memory alloy, respectively.

FIGS. 17A1, 17A2 and 17A3 show a memorized shape, in which FIG. 17A1 is a plan view, FIG. 17A2 is a front view, and FIG. 17A3 is a side view. FIGS. 17B1 and 17B2 show a shape obtained by deforming the FIG. 17A1 shape at a low temperature, in which FIG. 17B1 is a plan view and the FIG. 17B2 is a front view.

As shown in FIGS. 17A1, 17A2 and 17A3, the shape memory alloy fixating device for bone fractures includes a connector 71 of a wave form and a pair of holders 72 which can wrap a fracture portion with a circular ring or an elliptical ring.

As shown in FIGS. 17B1 and 17B2, the shape memory alloy fixating device for bone fractures is applied to a case that a shape of a bone is elliptical or rectangular after forming a low temperature phase, in which a connector 71 fixes the long axis of an ellipse or rectangle and a pair of holders 72 wrap the short axis thereof to thereby fix it.

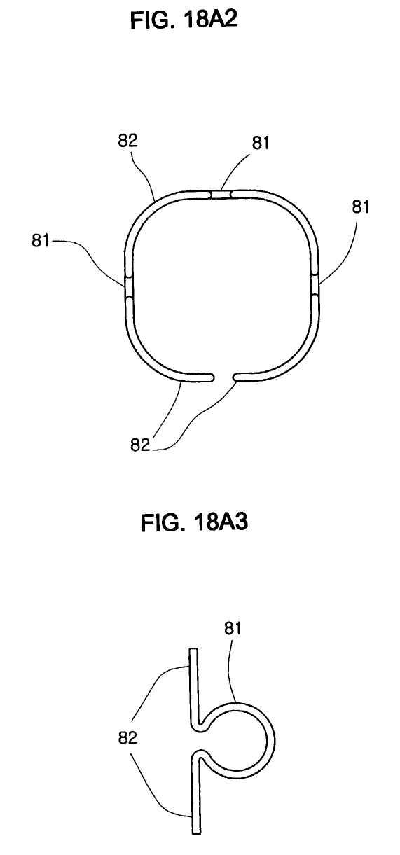

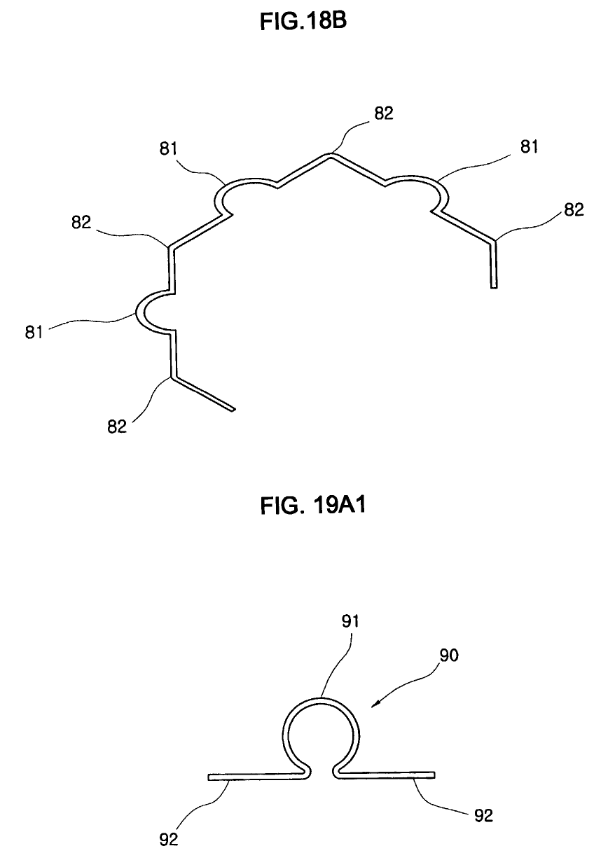

FIGS. 18A1, 18A2, 18A3, and 18B show a multi-omega ring type fixating device for bone fractures using a Ti—Ni—Mo shape memory alloy, respectively.

FIGS. 18A1, 18A2 and 18A3 show a memorized shape, in which FIG. 18A1 is a plan view, FIG. 18A2 is a front view, and FIG. 18A3 is a side view. FIG. 18B shows a shape obtained by deforming the FIG. 18A1 shape at a low temperature, so that the deformed shape can be easily applied to a fracture portion.

The multi-omega ring type fixating device for bone fractures shown in FIGS. 18A1, 18A2, 18A3, and 18B is same as that obtained by linking a plurality of the FIGS. 14A1, 14A2, 14A3, 14B1, 14B2 and 14C omega ring type fixating device for bone fractures. As shown in 18A1, 18A2, and 18A3, the shape memory alloy fixating device for bone fractures includes a plurality of rings 81 which can be fixed to a bone in the left and right directions, and a plurality of holders 82 which can wrap the bone in the left and right directions with respect to the rings 81.

As shown in FIG. 18B, the plurality of rings 81 and the plurality of holders 82 are widened laterally at a low temperature, to thereby wrap an elliptical fracture portion to easily fix it.

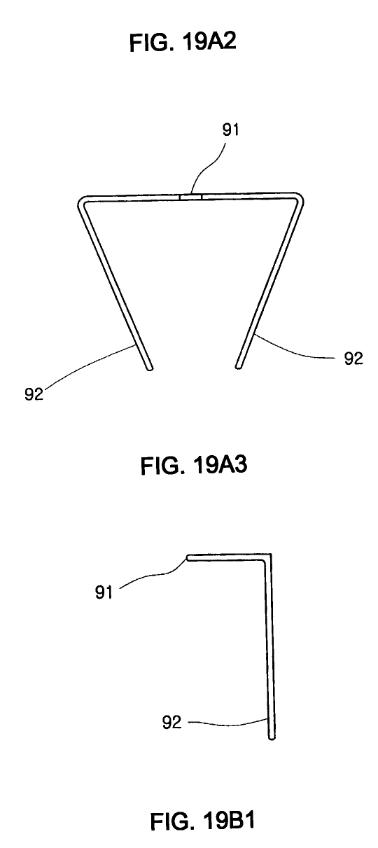

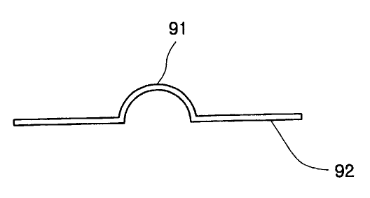

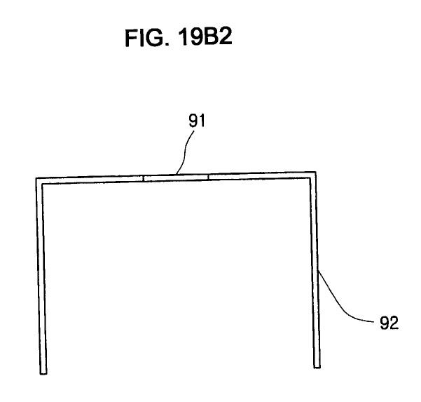

FIGS. 19A1, 19A2, 19A3, 19B1, and 19B2 show an omega type fixating device for bone fractures using a Ti—Ni—Mo shape memory alloy, respectively.

FIGS. 19A1, 19A2 and 19A3 show a memorized shape, in which FIG. 19A1 is a plan view, FIG. 19A2 is a front view, and FIG. 19A3 is a side view. FIGS. 19B1 and 19B2 show a shape obtained by deforming the FIG. 19A1 shape at a low temperature, in which FIG. 19B1 is a plan view and the FIG. 19B2 is a front view.

The shape memory alloy fixating device for bone fractures shown in FIGS. 19A1, 19A2 and 19A3 is same as the FIGS. 14A1, 14A2 and 14A3 shape memory alloy fixating device for bone fractures. A shown in FIGS. 19A1, 19A2 and 19A3, the shape memory alloy fixating device for bone fractures includes a ring 91 which can be fixed to a bone in the left and right directions and a holder 92 which can wrap the bone in the left and right directions with respect to the ring 91, in which the holder 92 is bent in the form of a triangle.

As shown in FIGS. 19B1 and 19B2, the ring 91 is widened laterally at a low temperature, and the holder 92 is open to thereby form a perpendicular plane with respect to the ring 91 and form a rectangle when viewed from the front.

The ring 91 pulls the holder 92 in the left and right directions to thereby reinforce a fixing force with respect to a fracture portion. Also, since a bone is hollow, the holder which is inserted to the center does not have any fixing force. As a result, the end portion of the holder is penetrated up to an opposite bone to be stably fixed.

If the holder does not penetrate a bone, a fracture portion of an opposite side tends to open due to an upper fixing force since the middle portion of the bone has no fixing force, which causes a plurality of clips driven into the bone.

Thus, the fixating device for bone fractures according to the present invention can be fixed to an opposite hard bone through which a pair of holders is inserted.

As described above, the Ti—Ni—Mo shape memory alloy according to the present invention can constantly maintain the transformation temperature even with a variation in an annealing treatment condition, can be obtained by adding Mo in a Ti—Ni alloy to enhance stability of R phase, and increase a corrosion resistivity to thus reduce Ni dissolution quantity.

Also, the fixating device for bone fractures using a shape memory alloy biomaterial can be applied to a living body very easily through a small-area incision and opening when compared with an existing surgical operation by features of the shape memory alloy. Also, the fixating device for bone fractures according to the present invention can be easily applied to the human body, to also shorten a surgical operation time and thus achieve an early recovery of patients.

The present invention is not limited to the above-described embodiments. It is apparent to one who has an ordinary skill in the art that there may be many modifications and variations within the same technical spirit of the invention.

What is claimed is:

1. A Ti—Ni—Mo shape memory alloy consisting of Ti of 48-52 at %, Ni of 48-52 at %, and Mo of 0.1-2.0 at % in a composition ratio.

2. The Ti—Ni—Mo shape memory alloy of claim 1, consisting of Ti of 51 at %, Ni of 48.5 at %, and Mo of 0.5 at %.

3. The Ti—Ni—Mo shape memory alloy of claim 1, consisting of Ti of 51 at %, Ni of 48.3 at %, and Mo of 0.7 at %.

4. A fixating device for bone fractures adopting the Ti—Ni—Mo shape memory alloy according to any one of claims 1 through 3, wherein part of said Ti—Ni—Mo shape memory alloy is incised, in order to form a single ring type.

5. The fixating device for bone fractures adopting the Ti—Ni—Mo shape memory alloy according to any one of claims 1 through 3, comprising: two holders worked in the form of a rod; and a connector which connects one end of each holder, in order to form a double ring type.

6. The fixating device for bone fractures adopting the Ti—Ni—Mo shape memory alloy according to any one of claims 1 through 3, comprising: a central circular ring; and a long leg formed by extending both ends of the ring and then crossing each other downwards from the ring, in order to form a long leg omega type.

7. The fixating device for bone fractures adopting the Ti—Ni—Mo shape memory alloy according to any one of claims 1 through 3, comprising a ring which can be fixed to a bone in the left and right directions and a circular holder which extended from both ends of the ring to thereby wrap the bone, in order to form an omega ring type.

8. The fixating device for bone fractures adopting the Ti—Ni—Mo shape memory alloy according to any one of claims 1 through 3, wherein wires are rolled and worked in the form of a rod, to then be formed as an ellipse type.

9. The fixating device for bone fractures adopting the Ti—Ni—Mo shape memory alloy according to any one of claims 1 through 3, wherein two wires are collected and widened in both sides to make a single wire, which includes an annular ring and a plate type holder which makes the wires twisted integrally or rolled, and said holder is bent into a triangle downwards from the ring, in order to form a clip type.

10. The fixating device for bone fractures adopting the Ti—Ni—Mo shape memory alloy according to any one of claims 1 through 3, comprising: a bent connector; and a pair of holders which are extended from both ends of the connector, to wrap a fracture portion in the form of a circle and an ellipse, in order to form a wave ring type.

11. The fixating device for bone fractures adopting the Ti—Ni—Mo shape memory alloy according to any one of claims 1 through 3, comprising: a plurality of rings which can be fixed to a bone in the left and right directions; and a plurality of holders which can wrap the bone in the left and right directions with respect to the rings, in order to form a multi-omega ring type.

12. The fixating device for bone fractures adopting the Ti—Ni—Mo shape memory alloy according to any one of claims 1 through 3, comprising: a ring which can be fixed to a bone in the left and right directions; and a holder which is extended in the left and right directions from the ring and bent as triangle downwards, in order to form an omega ring type.
